(19) United States
(12) Patent Application Publication
(10) Pub. No.: US 2014/0242600 A1
(43) Pub. Date: Aug. 28, 2014

(54) IMAGING THE HETEROGENEOUS UPTAKE OF RADIOLABELED MOLECULES IN SINGLE LIVING CELLS

(71) Applicants: Lei Xing, Palo Alto, CA (US); Colin Carpenter, Redwood City, CA (US); Peter Olcott, Stanford, CA (US); Guillem Pratx, Mountain View, CA (US); Conroy Sun, Sunnyvale, CA (US)

(72) Inventors: Lei Xing, Palo Alto, CA (US); Colin Carpenter, Redwood City, CA (US); Peter Olcott, Stanford, CA (US); Guillem Pratx, Mountain View, CA (US); Conroy Sun, Sunnyvale, CA (US)

(73) Assignee: THE BOARD OF TRUSTEES OF THE LELAND STANFORD JUNIOR UNIVERSITY, Palo Alto, CA (US)

(21) Appl. No.: 13/492,606

(22) Filed: Jun. 8, 2012

Related U.S. Application Data
(60) Provisional application No. 61/494,568, filed on Jun. 8, 2011.

(51) Int. Cl. G01N 33/50

(52) U.S. Cl. CPC G01N 33/5005
USPC 435/6.18; 435/14; 435/288.7

(57) ABSTRACT

A radioluminescence microscopy system and method for imaging the distribution of radiolabeled molecules in live cell cultures and tissue sections. Cells are grown and incubated with radiolabeled molecules on a scintillator plate or a scintillator plate is placed adjacent to the cells after incubation. Scintillation light produced by decay of radiolabeled molecules inside, bound to, or surrounding the cells, is recorded on an imaging device. Fluorescence microscopy of the same cells with other types of molecules of interest that are labeled with different fluorophores can be conducted concurrently and the biological activity of the labeled molecules can be correlated.

### CROSS-REFERENCE TO RELATED APPLICATIONS

This application claims priority from U.S. provisional patent application Ser. No. 61/494,568 filed on Jun. 8, 2011, incorporated herein by reference in its entirety.

### STATEMENT REGARDING FEDERALLY SPONSORED RESEARCH OR DEVELOPMENT

This invention was made with Government support under contract W81XWH-11-1-0087 awarded by the Department of Defense, under contract W81XWH-11-1-0070 awarded by the Department of Defense, under contract W81XWH-10-1-0506 awarded by the Department of Defense. The Government has certain rights in this invention.

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to cellular imaging, and more particularly to imaging radiotracer uptake in single living cells.

2. Description of Related Art

The visualization, characterization, and quantification of biological processes at cellular and subcellular levels are essential to understanding basic biology and disease. For example, fluorescence microscopy is an imaging technique where specific targets in a specimen are labeled with fluorescent molecules called fluorophores. When illuminated at a certain wavelength, fluorophores will emit light at a higher wavelength. By filtering out the excitation light, it is possible to visualize this fluorescent light using a microscope. However, fluorescence imaging has limited applicability beyond cell culture imaging and shallow tissue imaging due to the poor ability of the fluorescent light to penetrate biological tissue. Fluorophores are also relatively large molecules, which makes them unsuitable for studying some of the more subtle biological processes.

Radiolabeled molecules, also known as radiotracers or radiopharmaceuticals are typically biologically active molecules where at least one of the atoms is radioactive (radionuclide). For instance, ¹⁸F-fluorodeoxyglucose (FDG), a glucose analog tagged with a radioactive atom of fluorine, is a useful agent for visualizing cell metabolism and glycolysis. The radioactive atoms can be integrated into the structure of biological molecules without drastically changing their composition as occurs with fluorescent molecules. This allows the radioactively labeled molecules to pass as endogenous agents and move more easily across biological barriers.

The use of radiotracers to probe biological processes has several advantages over other approaches. Radiotracers are the preferred way to visualize biological processes deep within tissue in vivo. A radiotracer can also be synthesized with a chemical composition that is nearly identical to a given compound of interest. The concentration of radiotracers can be measured with exquisite sensitivity and their distribution can be imaged in vivo with positron emission tomography (PET) or single photon emission computed tomography (SPECT).

Radiotracers are used routinely in nuclear medicine for the diagnosis and treatment of diseases. Although radionuclide imaging with PET and SPECT is widely used to probe biological processes deep within tissues, little is known about the biological behavior of radiotracers at the individual cell level. Due to the low spatial resolution (1 mm at best), the smallest element that PET and SPECT techniques can resolve comprises approximately 10⁶ cells.

Autoradiography is another technique for imaging cellular uptake of radiolabeled molecules and is the standard method for imaging trace amounts of radiotracer in biological samples. In film autoradiography, a photographic film is placed in contact with a radioactive sample (cells or tissue sections) and is then exposed by the emission of energetic beta particles, typically from low-energy isotopes such as ³H, ¹⁴C, u and ¹²⁵I. The exposed film is then analyzed using light or electron microscopy. Film autoradiographs can be examined with optical or electron microscopy with sub-micron spatial resolution. However, film preparation is not compatible with the imaging of live cells. In addition, film autoradiography requires extremely long exposures due to poor detection efficiency and the procedure is only compatible with certain isotopes that have sufficiently low energy.

Digital autoradiography, using storage phosphor plates or direct detection, has higher detection efficiency and dynamic range but poorer spatial resolution (≧30 μm) that is insufficient to resolve individual cells. Furthermore, digital autoradiography lacks the capability of imaging the optical properties of the biological sample. Likewise, in vivo radiotracer imaging and scintillation counting can only measure signals from large cell populations.

Accordingly, current approaches for measuring radiotracer uptake in biological tissues are not capable of distinguishing single living cells. The averaging effect of measuring pooled cell populations masks important differences between cells belonging to the same population.

Therefore, there is a need to bridge the gap between the world of fluorescence microscopy and radionuclide imaging. There is a need for an imaging system that may be used to probe biological processes in a controlled cell culture environment, with the use of relatively small radiolabeled molecules. There is also a need for an imaging system that can image live cells with high spatial resolution with relatively short imaging times (minutes rather than days) and will avoid the disadvantages of autoradiography.

The present invention satisfies these needs, as well as others, and is generally an improvement over imaging systems in the art.

### BRIEF SUMMARY OF THE INVENTION

Radioluminescence microscopy bridges the gap between in vivo and in vitro imaging by enabling conventional radiolabeled compounds to be imaged in live cells with a spatial resolution that is sufficient to resolve single cells. With the invention, the same molecule can be imaged with high sensitivity in whole-body positron emission tomography (PET) scans in patients and in cell cultures. This feature may be useful for developing and characterizing new drugs and imaging agents. The invention can also be used in conjunction with standard fluorescence microscopy to correlate the activity of radiolabeled and fluorophore labeled molecules.

The radioluminescence microscope can also be employed to better understand how radiotracers are taken up by living cells under controlled experimental conditions. With increasing use of radiotracer imaging in research and in medicine, there is a need to better understand how properties that are specific to individual cells (e.g. gene expression, cell cycle, cell damage, and cell morphology) affect the uptake and retention of radiotracers. For instance, therapy and disease can alter cellular mechanisms in a heterogeneous manner and how these alterations affect radiotracer uptake at the single cell level is currently unknown and of critical importance in medicine and biology.

The present invention provides a radioluminescence microscope system and method that permits imaging of the cellular uptake and incorporation of radioactively labeled radiotracer molecules in individual living cells, for example. Using the appropriate set of optical filters, the system allows for the sequential acquisition of brightfield, fluorescence, bioluminescence, and radioluminescence images to correlate radiotracer labeling with other biomarkers such as gene expression, fluorescent probe binding, and cell morphology.

According to one embodiment of the invention, a radioluminescence microscope apparatus is provided that preferably has a deep-cooled electron-multiplying charge-coupled device (EM-CCD) camera, a high-numerical-aperture microscopy objective, an excitation light source, emission and excitation filters, and live cell culture instrumentation. The entire apparatus may be placed in a light-tight box to shield the sensitive camera from room lights. When the excitation light is blocked, the camera can record the light produced by energetic particles emanating from the decay of radiolabeled molecules inside the cell.

The cells may be grown directly on a transparent scintillator plate which is then placed in an imaging dish filled with cell culture medium. In a preferred embodiment, the scintillator plate is made from a non-hygroscopic, dense, high-Z material such as CdWO₄, fabricated into a thin plate (preferably less than 100 μm).

Initially, the scintillator plate is immersed in cell culture medium. After the cells have adhered to the surface of the scintillator plate and divided adequately, a radiolabeled molecule is added to the cell medium. The labeled molecules can be taken up by the cell, bind to a receptor, or be metabolized by a specific enzyme. After an incubation period, the scintillator plate, loaded with cells, is normally washed thoroughly to remove excess label and placed in a clean imaging dish with a thin glass bottom and clean cell culture medium is added prior to imaging.

In another embodiment, a special imaging dish is fabricated that has a thin bottom made from a transparent scintillator material, preferably CdWO₄. The cells are directly seeded and incubated in the special imaging dish. This method is advantageous because it allows the microscope objective to be placed in close proximity to the scintillator (i.e. without the need for a glass bottom), providing maximum light collection.

In another embodiment, a thin layer of a scintillator material is deposited on top of a thin glass substrate. Preferably, the thickness of the scintillator material is approximately 1 μm-10 μm and the thickness of the glass substrate is approximately 100 μm (for instance, #0 coverglass). The advantage of this embodiment is that a thinner layer of active scintillation material provides better spatial resolution since only the beginning of the beta ionization track results in the production of light. Furthermore, the layer of glass provides good mechanical strength.

In one embodiment, the invention can be used in conjunction with fluorescence microscopy. Live cells can be incubated with both radiolabeled molecules and with one or more molecules labeled with fluorescent dyes. Radioluminescence and fluorescence microscopy are then performed sequentially on the same cell culture. Because fluorescent dyes do not interact with the production and detection of scintillating light, the basic mechanism of imaging radiolabeled molecules is unchanged. Fluorescent labels are imaged by selecting the appropriate combination of excitation and emission filters in the filter wheel. Brightfield images can also be acquired by keeping the excitation and emission filters open. Radioactive, fluorescent and brightfield images are naturally co-registered and can be visualized on a computer as colored layers. Quantitative measurements can be performed on the images to estimate the total amount of radiolabeled molecules within a given cell.

Accordingly, an aspect of the invention is to image the cellular uptake of radiotracer labeled biologically active molecules at the single-cell level. The new technique could provide a quantitative determination of biological processes in individual cells under controlled conditions. Furthermore, correlation with biological parameters obtained from fluorescence and bioluminescence microscopy could provide new insight into the biological interactions of PET and SPECT radiotracers.

Another aspect of the invention is to provide a valuable tool for drug discovery, allowing radiolabeled drugs to be studied at multiple scales, first in a cell culture, then in a small-animal disease model, and, finally, in a patient cohort.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a schematic side view of a radioluminescence-fluorescence microscope imaging system according to an embodiment of the invention.

FIG. 2 is a detailed side sectional view of an imaging dish shown in FIG. 1.

FIG. 3: is a flow diagram for radioluminescence imaging according to the invention.

FIG. 4 is a flow diagram of an alternative method for radioluminescence imaging of cells with higher spatial resolution and improved quantitative accuracy.

FIG. 5A is a brightfield image of transduced HeLa cells and the accumulation of ¹⁸F-FHBG.

FIG. 5B is a radioluminescence image of the same field of cells shown in FIG. 5A.

FIG. 5C is a fluorescence image of the same field of cells shown in FIG. 5A.

### DETAILED DESCRIPTION OF THE INVENTION

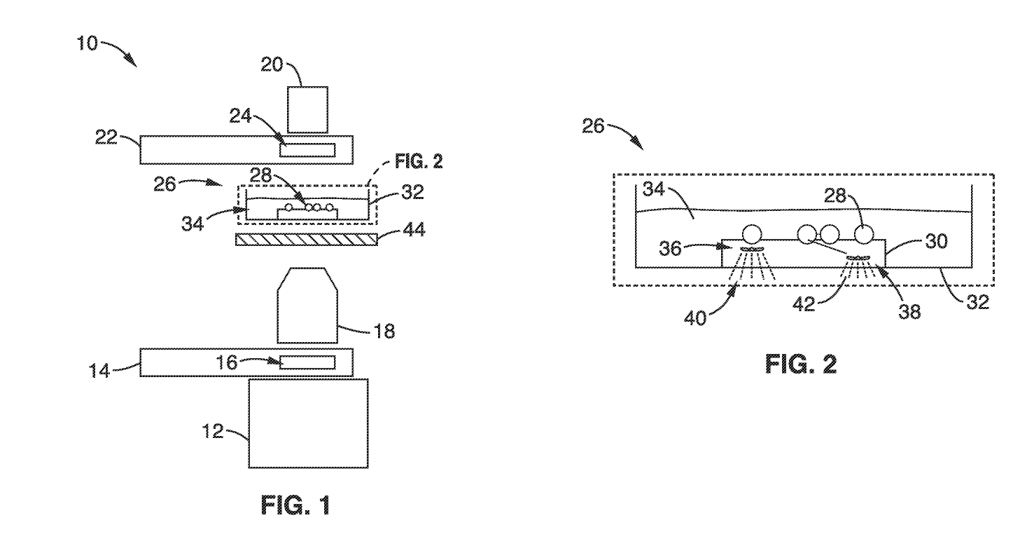

Referring more specifically to the drawings, for illustrative purposes the present invention is embodied in the apparatus and methods generally illustrated in FIG. 1 through FIG. 5C. It will be appreciated that the methods may vary as to the specific steps and sequence and the system architecture may vary as to structural details, without departing from the basic concepts as disclosed herein. The method steps are merely exemplary of the order that these steps may occur. The steps may occur in any order that is desired, such that it still performs the goals of the claimed invention.

Turning first to FIG. 1 and FIG. 2, an embodiment of an imaging system 10 according to the present invention is schematically shown in context of use. The illustrated apparatus is generally a microscope with a deep-cooled electron-multiplying (EM) or image-intensified (II) charge-coupled device (CCD) camera for high sensitivity, one or more microscopy objectives, an excitation light source, and emission and excitation filters. The microscope may also be equipped with temperature, humidity and CO₂ regulation for extended live cell imaging.

The entire apparatus is preferably placed in a light-tight box to shield the sensitive camera from room lights during image acquisition. When the excitation light is blocked, the camera can record the light produced by energetic particles emitted during the decay of radiolabeled molecules inside of, bound to, or surrounding the cells. The microscope is preferentially set-up in an inverted geometry, with the microscope objective underneath the sample to allow the use of a high-numerical-aperture oil objective. However, an upright microscope configuration may also be used for radioluminescence microscopy. The scintillation plate, on which live cells have adhered, may be placed such that the cells are either above or underneath the plate.

The preferred imager 12 is a deep-cooled electron-multiplying charge-coupled device (EM-CCD) camera. A typical imaging camera 12 is a 1024×1024 array of 13×13 μm pixels. The camera is preferably capable of both short and long image exposures. Exposure times of the camera may range from approximately 100 ms frames to approximately 30 minute frames. In addition, the imager 12 may also include a light intensifier to amplify the low amounts of light produced by individual beta decays and bring these weak signals above the intrinsic read noise of the camera. Suitable optical amplifying devices that are part of imager 12 include image intensifiers and electron multipliers, both coupled to the CCD camera imager 12. These devices have large, tunable gain and can amplify weak optical signals, such as those produced by a single beta decay.

The imager 12 is coupled to an emission filter wheel 14 that contains several emission filters 16 and a microscope objective 18, in the embodiment shown in FIG. 1. The emission filter wheel 14 and selectable emission filters 16 improve the signal to noise ratio of the acquired image by blocking any light outside of the range of the desired emitted wavelengths.

An excitation light 20, excitation filter wheel 22 with selectable excitation filters 24 are positioned opposite the objective lens 18 over the specimen and specimen platform 44. The excitation light 20 and filter 24 are used to control the illumination wavelength of light that enters the microscope. For example, the light source 20 in a fluorescence microscope setting will often produce a wide spectrum of light and an excitation filter 24 can be used to limit the illumination light to a specific wavelength range that is suitable for the particular specimen.

In the embodiment shown in FIG. 1 and FIG. 2, a live cell imaging scheme 26 is illustrated. The cells 28 are preferably grown directly on a scintillator plate 30 which is then immersed in an incubation dish filled with cell culture medium. In a preferred embodiment, the scintillator plate 30 is thin (less than 0.1 mm thick) and made from an inorganic scintillator that is a non-hygroscopic, dense, high-Z material such as CdWO₄. Preferably, tissue samples or cells are cultured on scintillator plates with dimensions of approximately 5 mm×5 mm×0.1 mm; however plates with any length and width dimensions suitable for the microscope selected can be used. Both sides of the scintillator plate 30 may be polished to allow for concurrent optical imaging. Thin scintillator plates of approximately 0.1 mm or less in thickness are preferred because they allow the short-working-distance objectives to focus on the upper side of the scintillator plate 30, where the cells are cultured.

After the cells have adhered to the surface of the scintillator plate 30 and divided adequately, radiolabeled molecules are added to the cell medium. The labeled molecules can be taken up by the cell 28, bind to a receptor, be metabolized by a specific enzyme or display some other biological activity. After an incubation period, the scintillator plate 30 that is loaded with cells 28 is taken out of its incubation dish and washed thoroughly. The plate 30 is preferably placed in a clean imaging dish 32 that has a thin glass bottom and has been filled with cell culture medium 34. The preferred thickness of the glass bottom of imaging dish 32 is approximately 100 μm to provide a clear image of the scintillator plate 30 and be sufficiently durable.

In an alternative embodiment, cells 28 are grown and incubated with radiolabeled molecules in a standard imaging dish 32. A scintillator plate 30 is placed in close proximity to the cells in the imaging dish 32 before imaging is performed.

In another embodiment, the scintillation plate 30 is made from a thin layer of a scintillator material that has been deposited on top of a thin glass substrate. The preferred thickness of the scintillator plate or layer of material on the substrate is approximately 1 μm to approximately 10 μm and the preferred thickness of the glass substrate is approximately 100 μm to provide good mechanical strength to the plate 30. The layer of scintillator material may also be deposited directly on the surface of the imaging dish 32. In these embodiments, a thinner layer of active scintillation material provides better spatial resolution since only the beginning of the beta ionization track will result in the production of light.

In another alternative embodiment, the imaging dish 32 is made from an appropriate scintillation material. The imaging dish 32 can be filled with cell culture medium 34 and the cells 28 can be grown directly in the imaging dish 32. The radiolabeled molecules can be added to the growth medium initially or after a period of cell growth in the scintillation imaging dish 32. In this embodiment, a scintillation plate 30 is not required since the function of the plate is performed by the bottom of the imaging dish.

With all of these embodiments, the imaging dish 32 is then placed on the sample platform or stage 44 of a microscope and the emissions from the cells 28 from the radiolabeled molecules are imaged.

Radioluminescence is the physical process by which the exposure of certain materials to ionizing charged particles produces measurable light. Due to the short range of beta particles (electrons or positrons), radioluminescence occurs near the location of the radioactive emitter. The range of these particles is further reduced in dense, high-atomic-number materials such as inorganic scintillators. Therefore, the radioactivity of individual cells 28 can be measured by seeding the cells sparsely onto a scintillator plate 30, and imaging the cells using a sensitive microscope with a high numerical aperture (NA) and high photon sensitivity.

Scintillator plates 30 are preferably made from dense inorganic crystals, with a high atomic number (Z) to minimize the range of alpha and beta particles, and a high light yield to maximize detection efficiency. The thickness of the scintillator plate 30 is also preferably minimized to reduce the negative impact of background gamma rays 38. One preferred material for scintillator plate 30 is cadmium tungstate (CdWO₄) because it has relatively high light yield (12,000-15,000 photon/MeV), high effective atomic number (Z_{eff}=64), high density (7.9 g/cm³), and no significant afterglow. Furthermore, CdWO₄ is non hygroscopic and can be used in contact with water.

Although cadmium tungstate is preferred, it will be appreciated that other scintillator materials may be used for radioluminescence microscopy, including but not limited to PbWO₄, BGO, GSO, LSO, LYSO, and Gd₂O₂S. Other materials may also be suitable for converting ionizing energy from radioactive decay particles near the cells into measurable signals. Some suitable materials can produce optical signals, such as inorganic, plastic, and liquid scintillators, and certain gases. Some other materials, such as semiconductors, can also convert the ionization into electronic charge that can be read out by an electronic circuit. Lastly, Cerenkov light is also produced when high-energy charged particles travel through matter. Cerenkov light can also be used to directly detect the radioactive decay, in which case no additional material is needed.

Scintillation light can be produced by two mechanisms. Most radioactive isotopes emit a combination of short range particles 36 (alpha and beta particles) and long-range gamma rays 38 as seen in FIG. 2. Long range annihilation photons and Bremsstrahlung X-rays can also be produced by short range, energetic particles. Short-range particles 36 generally produce a high resolution image of the cellular uptake, whereas long range particles produce a diffuse, undesirable background.

In another embodiment, a magnet or a magnetic coil in the microscope stage 44 is placed in the vicinity of the scintillator plate 30 to produce a strong magnetic field. Preferably, the magnetic field is oriented orthogonally to the plane of the scintillator plate 30. By curving the trajectory of emitted charged particles (such as electrons and positrons), the magnetic field can reduce the effective range of beta particles 36, thereby improving the spatial resolution.

The scintillation light cones 40 and 42, which contain information about the position and radioactivity of each cell 28, are magnified by a microscopy lens 18 and recorded by the camera 12. Exposure time controls the amount of light that is acquired, while the camera gain and the temperature controller help reduce the amount of read noise and dark noise, respectively.

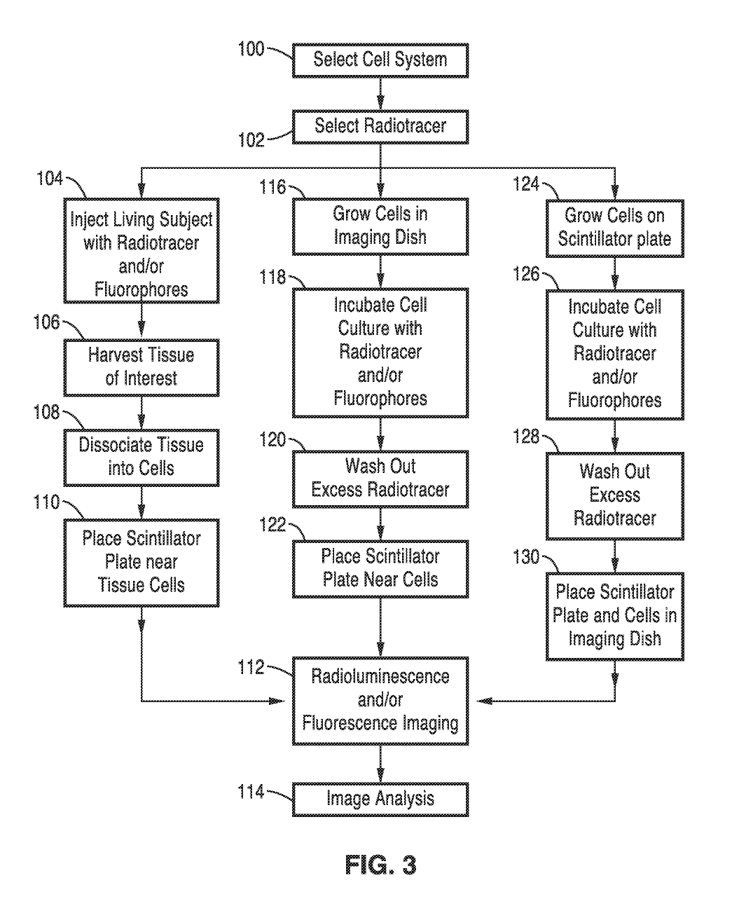

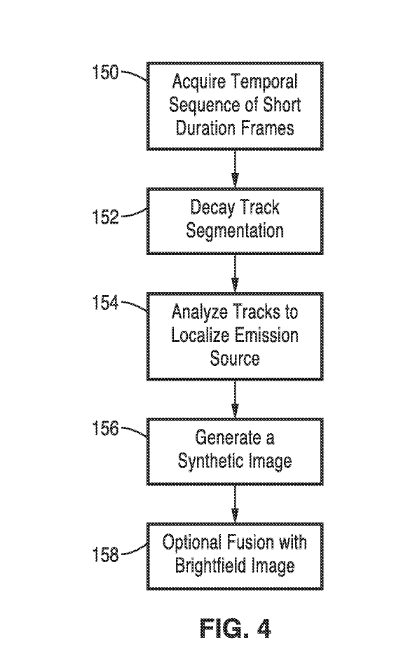

Referring also to FIG. 3 and FIG. 4, several methods for imaging the uptake and distribution of radiolabeled molecules in living cell cultures alone or concurrently with fluorescent labeled molecules are generally shown. It can be seen that the methods can be adapted to many different cellular systems with the use of many different tracer molecules that can be labeled in several different ways.

At block 100, the cellular system is selected for imaging. The cellular system may either be an immortalized cell line, or cells extracted from tissues from a living being. The biologically active molecule and radiolabel for evaluation are selected at block 102. The cell system and labeled molecules that are selected must be compatible. In one embodiment, one or more suitable fluorophores are also selected at block 102 for concurrent labeling of the subject molecule. This will allow concurrent radioluminescence and fluorescence imaging of the cells. In another embodiment, the cells have been genetically modified to express a fluorescent protein, which can also be concurrently imaged by fluorescence microscopy.

A wide variety of molecules, radiolabels and cellular systems can be selected at block 100 and block 102. For example, the molecule can be a small molecule, a protein fragment, or an antibody.

In one embodiment, the molecule that is selected at block 102 is an imaging agent for PET or SPECT imaging schemes. Examples of radiolabeled molecules suitable for imaging with radioluminescence microscopy include fluorodeoxyglucose (FDG), fluorothymidine (FLT), 5′-[p-(Fluorosulfonyl)benzoyl]guanosine (FSBG), and 9-(4-[¹⁸F]-fluoro-3-hydroxy-methylbutyl) guanine (FHBG).

Cells that have been selected may be prepared for imaging in several related ways as illustrated in FIG. 3. The tracer molecules that are selected at block 102 are labeled with appropriate radioactive labels and optionally fluorescent labels. Typical radioactive labels include ³H, ¹⁴C, ¹²⁵I, ¹⁸F, ¹³¹I, ¹¹C, ¹³N, ⁶⁸Ga, ³²P, ⁹⁰Y, ⁸⁹Zr, and ⁶⁴Cu. Typical fluorescent labels include organic dyes (e.g. Cy3, Cy5, FITC, TRITC, NBD, Texas Red, and fluorescein) as well as fluorescent proteins (e.g. GFP, RFP, and EYFP).

The selection of the cellular system and the type of radiolabeled molecule at blocks 100 and 102 will normally take into consideration the specimen preparation scheme that is desired. As seen in FIG. 3, the preparation of the cellular specimens may be initiated in a living subject or in a suitable cell line.

Initiation of cell labeling in a living subject is illustrated beginning at block 104 of FIG. 3. In many cases, the cellular system under study is an immortalized cell line. However, alternatively, the cells selected at block 100 can be obtained from a living multi-cellular organism that has been injected with a radiotracer. The cells are harvested by performing a biopsy, and dissociated by sectioning (frozen or parafilm section) or by trypsinization. In the embodiment shown in FIG. 3, the selected radiolabeled molecule is injected or otherwise delivered to the target tissue in a living being at block 104. The radiotracer selected and prepared at block 102 may also be injected together with molecules that are labeled with one or more types of fluorophores. These fluorescent molecules may target different biological processes so that the relationship between those processes and the uptake of radiotracer may be studied. Alternatively, these fluorescent molecules can be fluorescent proteins that are genetically expressed by the cells.

Since fluorescent dyes do not interact with the production and detection of scintillating light, the combination of fluorescent molecules with radiolabeled molecules does not interfere with the radioluminescence detection scheme. Radioluminescence and fluorescence microscopy can be performed sequentially on the same cell sample.

After an incubation time in the subject that is appropriate for the uptake and processing of the administered labeled molecules by the target tissue, the target tissue is sampled or harvested at block 106. The sampled tissue may then be disassociated into constituent cells and prepared for analysis at block 108.

The prepared cells at block 108 are preferably placed into an imaging dish and a scintillator plate is placed either below or above of the prepared cells at block 110. The imaging dish with the cell specimens and scintillator plate are placed into the imaging microscope for radioluminescence microscopy and/or fluorescence imaging at block 112. The scintillation light is produced by the decay of radiolabeled molecules that are inside, bound to, or surrounding the cells and the scintillation light is preferably recorded by the imaging device and imaged at block 112. The recorded images are then analyzed at block 114.

In one embodiment, the tissue is sliced into thin sections using standard methods such as frozen sectioning or paraffin sectioning. The thin tissue section may be pressed between a scintillator plate and a glass-bottom imaging dish, or between two scintillator plates at block 110. The imaging dish is then placed into an imaging microscope and imaged at block 112. Scintillation light from radiolabeled molecules in the tissue section produce light that is visualized by the imaging microscope. Standard tissue staining and fluorescence imaging can be performed concurrently to correlate radiotracer uptake with other biological markers.

Individual cells such as those from various immortal cell lines can also be selected at block 100 as a cellular system. At block 116 of FIG. 3, the cells selected at block 100 are grown in an imaging dish under suitable conditions. Cells grown in the imaging dish can then be incubated with radiolabeled molecules prepared at block 102 for a period of time. Incubation times at block 118 may vary depending on the nature of the labeled molecules and the type of cells selected and the biological system that is being investigated. The radiolabeled molecules prepared at block 102 may also be labeled with a fluorophore. In this embodiment, the radioluminescence imaging methods can be used in conjunction with fluorescence microscopy.

Live cells can also be incubated with both radiolabeled and fluorescent molecules at block 118 simultaneously or in sequence. The most common use of fluorescent/radioluminescence imaging may be the use of fluorophore labeled molecules that target a process that is different from the process targeted by the radioactive molecule. For example, the radioactive labeled molecules could target glucose metabolism while the fluorescent molecules target cellular proliferation. This allows the study of the relationship between the two different processes. A second type of fluorophore can label a third type of molecule and the activity of the third molecule can be correlated and compared with the others.

The excess radiotracer and fluorophore labeled molecules in the cell culture media are preferably removed from the cell culture media at the conclusion of the incubation period at block 120. The old cell culture media with the excess label is preferably replaced with clean media at block 120.

The incubated cells are then placed in contact with, adjacent to or in close proximity to a scintillator plate at block 122 and placed in the imaging device for imaging at block 112.

In an alternative embodiment, the cells are grown directly on a scintillator plate that has been immersed in cell culture medium at block 124. Alternatively, the cells are grown at block 124 in a special imaging dish that has been fabricated with a thin bottom made from a transparent scintillator material or a thin layer of scintillator material, preferably CdWO₄. The cells are directly seeded and incubated in the scintillator imaging dish. This embodiment allows the microscope objective to be placed in close proximity to the scintillator (i.e. without the need for a glass bottom) in order to maximize the light collection.

Preferably, the cells are grown on the scintillator plate with a low degree of confluency to ensure sufficient separation between cells. Typically, a separation of 10 μm between cells is sufficient for the reliable estimation of radiotracer uptake in individual cells. To achieve a low degree of cell confluency, a small number of cells must be initially seeded on the plate. For instance, 10,000 MDA-MB-231 cells can be seeded onto a 5 mm×5 mm scintillator plate. Because the cells attach randomly to the plate, not all cells will satisfy the separation requirement. Other possible approaches may be used to place the cells on the scintillator plate according to the desired arrangement. Indentations may be fabricated in the plate for cells to attach more easily at certain given locations on the plate. Alternatively, the cells may be placed in contact with the plate via the action of a microfluidic system, capable of precisely positioning the cells within a narrow channel.

At block 126, the radiolabeled molecules or the fluorophore labeled and radiolabeled molecules are introduced into the cell culture medium and the cells and radiolabeled molecules are incubated for an appropriate period of time to allow the uptake of the selected and labeled molecules by the cells.

The excess labeled molecules in the cell culture media is preferably removed and replaced with clean cell culture media at block 128. Optionally, the cell culture media can be added and removed several times to further wash out the excess labeled molecules. The scintillator plate with the live labeled cells can then be placed in an imaging dish at block 130 and then imaged at block 112.

In an alternative embodiment, cells can be grown on a scintillator plate immersed in a cell culture medium at block 124 and the scintillator plate is placed in an imaging dish after the cells have grown for a suitable time on the scintillator plate. Radiolabeled molecules are introduced into the cell culture medium. The concentration of radiolabeled molecules in the cell medium is changed over time. Since the cells are alive, they respond to the changing concentration of radiolabeled molecules in the cell medium. Imaging at block 112 is performed at multiple time points to visualize and measure the changes over time in response to varying concentrations of labeled molecules.

In another embodiment, the cells that have been incubated with labeled molecules are fixed prior to radioluminescence or fluorescence imaging at block 112. Rather than dynamic imaging with live cells, static imaging and measurements are taken from the cells at a fixed time point. Imaging of single living or fixed cells can also be performed with high-energy radionuclides (with beta energy preferably higher than 250 keV), such as those radionuclides used for PET and SPECT imaging (¹⁸F, ⁶⁴C, ⁸⁹Zr, ¹²⁴I, ¹²³I, etc.).

Accordingly, the radioluminescence microscopy methods can be used alone or in conjunction with fluorescence microscopy. As seen in FIG. 3, the combined approach of radioluminescence microscopy and fluorescence microscopy can be conducted with cells obtained from a living subject or cells from cell culture. The selected biologically active molecules under examination are normally both radiolabeled and fluorescence labeled. However, it is also desirable to radiolabel one type of tracer molecule and fluorescence label another type of molecule and incubate both molecules simultaneously or sequentially with the cells as shown in FIG. 3, in another embodiment.

In the combined approach, imaging at block 112 with radioluminescence and fluorescence microscopy is preferably performed sequentially on the same cell culture field. Generally molecules labeled with fluorescent dyes are imaged at block 112 by selecting the appropriate combination of excitation and emission filters in the filter wheel of the imaging microscope. Brightfield images can also be acquired by keeping the excitation and emission filters open. Radioactive, fluorescent and brightfield images are naturally co-registered in this embodiment. The resulting scintillation and fluorescent images can be analyzed at block 114. For example, quantitative measurements can be performed on the images to estimate the total amount of radiolabeled molecules within the cell. A region of interest can be drawn around each cell. The radioluminescence signal can be summed over each region of interest to estimate the amount of radiotracer present in the underlying cell, preferably using a calibration procedure.

Radiotracer molecule uptake can also be correlated with other biological markers measured with fluorescent molecules. For instance, the uptake of FDG can be correlated with fluorescent markers of hypoxia and cell proliferation. The cellular heterogeneity of radiotracer uptake may also be observed at the single cell level.

In another approach, harvested cells (or cells incubated with a radiolabeled molecule) are injected into a liquid flowing near a scintillator plate, so the radioactivity of each individual cell is measured precisely. This approach can be used in conjunction with flow cytometry to correlate the amount of radiolabeled molecule linked to the cell with other standard markers. Microfluidics technology may be used to flow such a liquid near a scintillator.

Turning now to FIG. 4, an alternative embodiment of the radioluminescence microscopy system and method is schematically shown. This extension of radioluminescence microscopy is referred to as super-resolution radioluminescence microscopy because it aims to resolve cellular uptake of radiotracer molecules beyond the spatial resolution limit imposed by the physical propagation of the beta particles through the scintillator plate.

Radioluminescence microscopy may suffer from a few physical limitations in some settings. First, the energetic beta particles can propagate up to 100 μm within the scintillator, producing a track of optical photons. This effect can degrade the ability of the radioluminescence microscope to localize radioactive uptake within cells since the signal is spread out along a long track. Secondly, because each beta particle is emitted from the cell with a variable amount of energy, each decay event produces a variable amount of light. This effect can increase the variability of the signal measured by the radioluminescence microscope and reduces its quantitative accuracy.

In order to account for these two physical limitations, image acquisition is performed not as a single long frame but rather as a sequence of many short frames. While a 5 minute frame can accumulate light from thousands of beta decays, a 100 ms frame only captures a few beta decay events. Provided that these beta decays are well separated and do not spatially overlap, they can be resolved and analyzed automatically by custom image processing software. The information obtained from such analysis can be used to produce a synthetic image with higher spatial resolution and improved quantitative accuracy.

Accordingly, at block 150 of FIG. 4, a temporal sequence of short duration frames is acquired. Many short frames are preferably acquired using high-gain cameras to capture the small optical signals emitted by single beta particles. An optical amplifier may also be used to improve the acquired frames at block 150.

Decay tracks are segmented at block 152. For example, a beta track segmentation can be performed as follows: Each short frame is filtered by a Gaussian kernel and transformed using a H-maxima Transform, which removes local maxima that are not substantially higher than their surroundings. The resulting grayscale image can then be thresholded to produce a binary image. Morphological operations may be applied to fill in holes and suppress small connected components. Finally, the connected components are identified and labeled with an index. Each connected component corresponds to a single beta track.

At block 154 of FIG. 4, the beta track is analyzed to localize the emission source. One preferred method for automatically analyzing a beta track at block 154 is the following classification scheme. Once the entire track has been segmented, an algorithm preferably classifies it as “short”, “long,” or “out-of-focus.” A “short” track exhibits a concentration of the optical energy over a small area. For these tracks, the beta emission location can be estimated by maximizing the optical signal and the proximity to a cell, since it is more likely that the beta particle was emitted from a cell. Indeed, extracellular radiotracer is distributed uniformly within the cell medium, and is not expected to be in close proximity to the scintillator.

A “long” track is typically produced by a very energetic beta particle emission. These long tracks can be more challenging to analyze, since the beta particle travels far away from its emission location. The long tracks are preferably analyzed by an iterative procedure. The starting point is chosen as the location on the track with the highest optical intensity. Then, the track is traced in one direction. The algorithm first moves by a constant step length in the direction that maximizes the total optical signal that is computed by integration over the step length. As long as the optical signal remains above a certain threshold, the iterative algorithm continues and searches for the next direction. The search is limited to the unexplored half plane to prevent the algorithm from choosing a direction that would bring it back to its starting point. Furthermore, large deflection angles are discouraged by a numerical penalty function since these are physically unlikely.

After the track has been traced to its end, the second half of the track is traced by resetting the algorithm to the starting position, but tracing the track in the opposite direction. After the second half of the track has been traced, the algorithm must orient the track, that is, find which end of the track corresponds to the beta emission location and endpoint, respectively. This can be achieved by taking into account proximity to a cell, the thickness of the track or the intensity of the optical emission.

The “out-of-focus” beta tracks are discarded. For the most part, these tracks correspond to gamma interactions that occurred deep within the scintillator plate, far from the focal plane. Gamma photons do not contribute any useful information to the signal but add an undesirable background. Therefore, the super-resolution approach has the additional advantage that these undesirable events can be filtered out from the final image.

After all the beta tracks have been segmented, analyzed and localized, a synthetic image can be produced at block 156 by locating each presumed beta emission. Such image is the super-resolution image, in which the blurring inherent to the physical propagation of the energetic betas has been removed. The synthetic image produced at block 156 can be obtained without any prior information or requiring prior knowledge of the cell boundaries. In one embodiment, a segmented mask obtained from a brightfield image can be used to favor the localization of beta decays near a cell. However, any other method for localizing the cell boundaries may be used.

Optionally, at block 158, the synthetic image that is produced at block 156 can be compared with fluorescence results by fusing the image over a brightfield image that has been obtained from the same cells.

Embodiments of the present invention may be described with reference to flowchart illustrations of methods and systems according to embodiments of the invention, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

The invention may be better understood with reference to the accompanying examples, which are intended for purposes of illustration only and should not be construed as in any sense limiting the scope of the present invention as defined in the claims appended hereto.

### Example 1

To visualize the uptake of radiotracer at the microscopic level, cells were cultured directly on a scintillating plate made of a material that converts incident beta radiation into optical photons via radioluminescence. In these experiments, scintillation plates were used with dimensions 10 mm×10 mm×0.5 mm that were made of CdWO₄, a dense, high-Z, non-hygroscopic material, with both sides polished to allow for concurrent optical imaging (MTI Corp.). In one experiment, HeLa cells were seeded and cultured on the scintillating plate, immersed in cell culture medium (Dulbecco's Modified Eagle Medium containing 10% fetal calf serum) for 12-18 hours. After the cells had adhered to the surface of the scintillator plate and divided adequately, they were fasted for two hours in glucose-free cell medium and incubated for 20 minutes at 37° C. with 400 μCi of ¹⁸F-fluorodeoxyglucose (FDG). The plate, loaded with cells, was then washed thoroughly and placed in a 100 μm-thin glass-bottom microscopy dish (FD35, World Precision Instruments) that had been filled with fresh cell culture media. the imaging dish was placed in a bioluminescence microscope (LV200, Olympus) outfitted with a 40×, 1.4 numerical aperture, oil lens (UPLFLN40XO, Olympus) and a deep-cooled electron-multiplying charge-coupled device (EM-CCD; ImageEM C9100-14, Hamamatsu). The C9100-14 is a back-thinned frame transfer CCD, with a 1024×1024 array of 13×13 μm pixels. The LV200 is also equipped with live-cell imaging capabilities, including temperature, humidity, and % CO₂ regulation for time-lapse acquisitions. Brightfield images were acquired with no EM gain, and both excitation and emission shutters open.

Radioluminescence images were taken with an EM gain of 251/1200, the excitation shutter closed, and the emission shutter open. Due to the short working distance of the microscope objective (200 μm), the scintillating plate was placed up-side-down on top of the cells. To optimize radioluminescence image focus, the microscope was first focused on the cells in brightfield mode. Then, in luminescence mode, the image sharpness was optimized by varying the focal distance while acquiring 5 minute-exposure images. It was found that the best radioluminescence focus was achieved when the cells displayed slightly-blurred negative contrast in the corresponding brightfield image. The experiments were concluded by acquiring 20 minute exposure images. The HeLa cell experiment showed good colocalization between the cell outline, seen in the brightfield image, and the radioluminescence intensity.

### Example 2

To further evaluate the performance of the imaging set-up, a drop of FDG (activity≦2 μCi) was placed between the imaging dish and the scintillating plate. Upon evaporation of the water solvent, FDG precipitated into small solid deposits that could be seen on both brightfield and radioluminescence images. The size of these deposits was measured by fitting them with 2-D Gaussian functions.

Good correlation (ρ=−0.79) was observed between brightfield and radioluminescence images. A magnified view reveals weaker features present in both imaging modes. A particularly intense deposit was selected and measured by fitting with an isotropic 2-D Gaussian. The full width at half maximum (FWHM) was found to be 5.0 μm for the brightfield image and 6.9 μm for the radioluminescence image, yielding an estimated system resolution of 4.7 μm (FWHM).

### Example 3

In another experiment, the sensitivity of the system was evaluated by imaging the decay of 2.6 μCi of FDG over 24 hours. A small drop of FDG was mixed with glycerol and placed on an imaging dish. The mixture was then heated for several hours to allow the water solvent to evaporate, thereby ensuring that no water would evaporate during the acquisition. It was verified that the mixture was uniformly spread between the scintillator plate and the imaging dish. The mixture was imaged every 31 minutes, in 30 minute-long frames, with an EM gain of 251/1200. Within a large (370000 pixels) region of interest, pixel values were expressed as a percentage of their value in the first frame. The mean pixel value and the range of pixel-to-pixel fluctuations—defined by one standard deviation—were computed for each frame. For a quantitative assessment of radioluminescence intensity, flat-field and dark image corrections were applied to all acquired images.

Initially, 69.7 fCi of FDG per CCD pixel were present in the field-of-view. As the activity decayed, the aggregate radioluminescence signal, obtained by summing 370000 pixels, decayed at a constant rate down to 1 fCi per pixel. However, individual pixels were subject to much stronger noise, as evidenced by the pixel-to-pixel variability that was observed. It was estimated that the detection threshold was 10 fCi per pixel, which is the amount of activity required to achieve a signal-to-noise ratio (SNR) of 5.

From the foregoing it can be seen that radioluminescence microscopy is a promising new approach for imaging the binding and uptake of a radiotracer in living cell cultures. For the first time, it is shown that it is possible to visualize how individual cells uptake radiotracers using only off-the-shelf equipment. In the future, this information could be correlated with fluorescence and bioluminescence biomarkers, leading to new insights into how biology affects the specificity of PET and SPECT tracers. Using a calibration curve, quantitative measurements of radiotracer concentration within individual cells can be obtained, provided that corrections for field flatness, photon background, and dark signal are applied.

### Example 4

Radiotracers play an important role in interrogating molecular processes both in vitro and in vivo. However, current methods are limited to measuring average radiotracer uptake in large cell populations and lack the ability to quantify cell-to-cell variations as a result. To further demonstrate the apparatus and method for visualizing radiotracer uptake in single living cells, radioluminescence microscopy was used in conjunction with standard fluorescence microscopy. In this illustration, the common radiotracer [¹⁸F]fluorodeoxyglucose (FDG) was used. FDG is preferentially taken up and retained within tissues with high glucose metabolism such as found in malignant tumors. Therefore, measuring FDG uptake in a heterogeneous cell population is of great interest as it may help better understand the heterogeneous metabolic alterations displayed by tumors, and the impact that the tumor microenvironment has on these alterations. The radioluminescence microscopy set-up consisted of cells adhering to a 100 μm-thin CdWO₄ scintillator plate that was immersed in a glass-bottom dish filled with cell culture medium as shown schematically in FIG. 2. The dish was imaged using an inverted microscope fitted with a high-NA objective and an electron-multiplying charge-coupled device (EM-CCD).

Brightfield images were acquired with no EM gain, a neutral-density filter on the excitation, and the emission shutter open. Radioluminescence images were taken with a 40× magnification objective, an exposure time of 5 min, an EM gain of 251/1200, 2×2 pixel binning, the excitation shutter closed, and the emission shutter open. The brightfield mode was used to set the microscope into focus. Optimal radioluminescence focus was achieved when the cells displayed sharp positive contrast in the corresponding brightfield image. For fluorescence microscopy, a 460 nm/535 nm filter set for 2-NBDG imaging (Chroma, D460/50x and D535/40m) and a 540 nm/600 nm filter set for RFP imaging (Chroma, HQ540/40x and HQ600/50m) were used.

Samples of MDA-MB-231 human breast cancer cells were obtained and cultured in Leibovitz's L15 medium supplemented with 10% fetal bovine serum. One side of the scintillator plate was coated with fibronectin (10 μg/ml) to allow the cells to attach. After the plate had dried, the cells were seeded by placing a 50 μl drop containing 10⁴ cells on to the fibronectin-coated plate.

To investigate the uptake of FDG by single cells at a fixed time-point, human breast cancer cells (MDA-MB-231) were deprived of glucose for 1 hour, then incubated for 1 hour at 37° C. with FDG (400 μCi) and 2-[N(7-nitrobenz-2-oxa-1,3-diaxol-4-yl)amino]-2-deoxyglucose (2-NBOG; 100 μM), a fluorescent glucose analog. After washing the cells, brightfield, radioluminescence and fluorescence micrographs were acquired.

To measure radiotracer uptake in single cells, circular regions of interest (ROI's; diameter, 24 μm) were manually placed on the cells using the brightfield micrograph. Similar ROI's were placed in the background as controls. Cell radiotracer uptake was defined as the mean pixel intensity within the ROI of the corrected radioluminescence image. The same procedure was also applied to fluorescence micrographs.

Good co-localization between the radioluminescence intensity and the cell outline was observed on brightfield images. Furthermore, the radioluminescence intensity varied significantly from cell to cell, indicating heterogeneous uptake of FDG correlated with uptake of 2-NBDG (P<10⁻⁵, r=0.74). An exact correlation between FDG and 2-NBDG was not expected due to (1) possibly distinct transport mechanisms; and (2) the inability of 2-NBDG to fluoresce once it has been metabolized. A line profile through the fluorescence and radioluminescence images confirmed co-localization of FDG and 2-NBDG.

### Example 5

The transport and retention of FDG in a cell is influenced by multiple factors, such as the expression of various genes, the density of glucose transporters on the cell surface, the cell size, and the levels and activities of hexokinase and phosphatase enzymes. Under steady-state conditions, the intra- and extracellular FDG concentrations are in equilibrium. However, rapid changes in the extracellular environment induce a transient response characteristic of the cell's glucose metabolism parameters. These parameters can be estimated using pharmacokinetic modeling techniques. The ability to manipulate a cell's environment is unique to an in vitro setting and cannot be easily replicated in vivo.

Furthermore, pharmacokinetic modeling from PET or gamma counting measurements requires assumptions such as uniform radiotracer concentration and homogeneous rate parameters for each compartment. These assumptions may not be satisfied in practice because each cell in the compartment is characterized by unique parameters. For accurate characterization of cellular parameters, pharmacokinetic modeling should be performed at the level of a single cell.

To investigate the utility of radioluminescence microscopy for single-cell pharmacokinetic studies, FDG uptake, FDG efflux and FDG withdrawal conditions were analyzed. FDG uptake in breast cancer cells (MDA-MB-231) over time was monitored by depriving cells of glucose for 1 hour and then adding 5 μCi of FDG to the medium. Serial brightfield and radioluminescence images were acquired every 6 minutes for a period of 8 hours. Although FDG uptake varied significantly from cell to cell, all cells displayed a linear increase in radioactivity, followed by a plateau and a slow decrease after 3 hours.

To monitor FDG efflux, breast cancer cells (MDA-MB-231) were subjected to conditions known to minimize FDG influx, i.e. thorough competition from glucose and withdrawal of FDG. The addition of glucose to the medium (25 mM) at 2 hours lead to a decline in cell radioactivity as FDG and glucose competed for the same glucose transporters. Similarly, withdrawal of FDG from the media of cells that had previously been incubated with FDG (400 μCi, 1 hour) also resulted in a fast decrease in cell radioactivity.

The influx of FDG into glucose-deprived cells was described using the following two-tissue compartmental kinetic model:

$$\frac{C ( t )}{{C}_{a}} = \frac{{K}_{1} {k}_{2}}{{( {k}_{2} + {k}_{3} )}^{2}} ( 1 - {e}^{- ( {k}_{2} + {k}_{3} ) t} ) + \frac{{K}_{1} {k}_{3}}{{k}_{2} + {k}_{3}} t$$

where Cₐ is the extracellular FDG concentration (assumed to be fixed); C(t) is the time-dependent intracellular FDG concentration (including free FDG and bound FDG-6-P); and K₁, k₂ and k₃ are the rate constants representing influx, efflux, and irreversible phosphorylation of FDG, respectively. Fort>>1/(k₂+k₃, the intracellular and extracellular compartments are in equilibrium, and the intracellular concentration of FDG increases linearly with time due to irreversible trapping of FDG. The parameters of this linear rise (i.e. slope and intercept) are the Patlak coefficients. The slope of the linear rise is the product of two terms, namely K₁ the influx rate, and k₃/(k₂+k₃) is the fraction of the intracellular FDG irreversibly metabolized. A non-linear weighted least-squares fitting was used to estimate the parameters of the model. The fitting weights were adjusted to decrease the contribution of later time points, which have higher noise due to radioactive decay.

Large variations in the Patlak coefficients were found across the cells that were imaged indicating that seemingly identical cells metabolize glucose heterogeneously. This finding suggests that the cells within a tumor are not represented equally in an FDG-PET scan; rather, a small number of cells contribute the majority of the PET signal. Also, the majority of cells stopped accumulating FDG at approximately 3 hours.

A mathematical model was also derived to represent FDG efflux from a cell. The model assumed that (1) following withdrawal of FDG, the concentration of FDG in the cell culture medium remained negligible due to the large extracellular volume (0.2 ml), and (2) k₃>>k₄. Under these assumptions, FDG concentration within a single cell can be described by the sum of a slow and a fast exponential decay.

Efflux of FDG from cells was modeled using a two-tissue compartmental model:

C(t)=a₁e^{-λ}^{₁}^{t}+a₂e^{-λ}^{₂}^{t}

where a₁ and a₂ are positive coefficients that depend on the initial conditions, and λ₁ and λ₂ are the eigenvalues of the differential system of equations describing transport of FDG between compartments. The rate constant k₄ which models the possible dephosphorylation of FDG-6-phosphate (FDG-6-P) was included in this model but assumed to be much smaller than k₃. Furthermore, due to the large extracellular volume (0.2 ml), the concentration of FDG in the cell culture medium was assumed to remain negligible after withdrawal of FDG. Under these assumptions, the eigenvalues can be approximated as

$${λ}_{1} = {k}_{2} + {k}_{3} + \frac{{k}_{3} {k}_{4}}{{k}_{2} + {k}_{3}}$$

$$and$$

$${λ}_{2} = \frac{{k}_{2} {k}_{4}}{{k}_{2} + {k}_{3}} .$$

These rate parameters were estimated by fitting the efflux model to the measured time-activity curves. For cells for which the solution of the fit yielded λ₁≈λ₂ or λ₂<1 min⁻¹, the efflux curve was fitted with a single exponential function. In the special case of irreversible trapping (k₄=0), the model is described by a single exponential decay with rate: λ₁=k₂+k₃. The model was in agreement with radioluminescence measurements of single cells, confirming that two processes are occurring concurrently at different rates. Unbound FDG contained within the cell quickly diffuses out following concentration gradients, whereas FDG-6-phosphate (FDG-6-P) requires slow dephosphorylation to cross the cell membrane.

### Example 6

To further exploit the microscope's ability to visualize both fluorescent and radioactive probe distributions, human cervical cancer cells (HeLa) were transduced with a dual fusion reporter vector, comprising reporter genes encoding the monomeric red fluorescent protein (mrfp1) and the mutant herpes simplex virus type 1 truncated thymidine kinase (HSV1-ttk). HSV1-ttk can selectively metabolize and trap radiolabeled substrates such as FHBG. Substrates such as 9-(4-[¹⁸F]Fluoro-3hydroxymethylbutyl) guanine (¹⁸F-FHBG) have low affinity for mammalian thymidine kinase (TK) enzymes but high affinity for HSV1-TK. FHBG is selectively metabolized and trapped within transduced cells so it can be used to image cell trafficking in living subjects with PET.

PCR amplification and standard cloning techniques were used to insert the mrfp and ttk genes from plasmid pCONA3.1-CMV-hrl-mrfp-ttk. Lentiviral EF1-gfp vectors were obtained and the gfp fragment was removed from the vector and replaced by mrfp-ttk. For PCR amplifications, different 5′ and 3′ end primers were used to generate the fusion vector (EF1-mrfp-ttk).

HeLa human cervical cancer and 293T human embryonic kidney cells were acquired and cultured in high-glucose Dulbecco's modified eagle medium supplemented with 10% fetal bovine serum. 293T cells were used to produce the lentivirus following standard procedures. HeLa cells were transduced with concentrated lentivirus for 48 hours and then trypsinized and seeded directly onto a scintillator plate coated with fibronectin (10 μg/ml), one day before imaging.

For imaging of cell transduction with FHBG and RFP, transduced HeLa cells were incubated for 2 hours with 300 μCi of ¹⁸F-FHBG. Radioluminescence microscopy of transduced cells incubated with FHBG demonstrated focal radiotracer uptake, with individual cells clearly resolvable under 100× magnification.

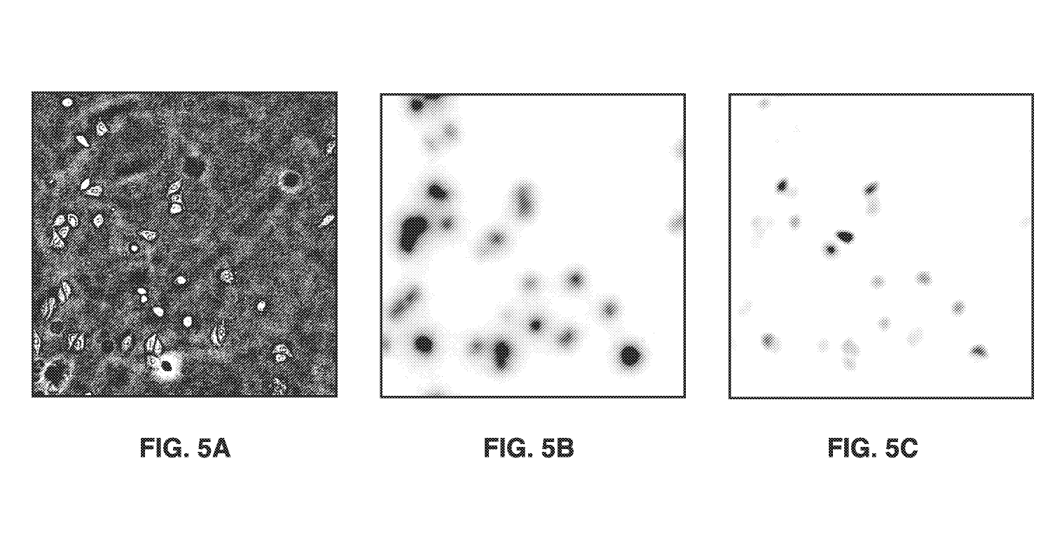

In transduced cells, accumulation of FHBG was coincident with expression of mrfp1 measured by fluorescence microscopy. A brightfield image (FIG. 5A), a radioluminescence image (FIG. 5B) and a fluorescence image (FIG. 5C) were obtained from the same field. It was seen that 88% of the cells visible on the brightfield image (217/245) were clearly distinguishable both on radioluminescence (FIG. 5B) and fluorescence images (FIG. 5C), while only 9% (21/245) could not be seen on either image. The remaining 7 cells were excluded from the analysis due to ambiguous radioluminescence intensity caused by the proximity to a strongly positive cell.

Generally, radioluminescence signals for FHBG-positive and FHBG-negative cells were more distinctly separated than fluorescence signals for RFP-positive and RFP-negative cells. While uptake of FHBG was coincident with RFP fluorescence signal, fluorescence intensity was not strongly predictive of radioluminescence intensity (r=0.34), indicating that although HSV1-tk reporter gene expression is required for FHBG uptake, the level of gene expression is not solely responsible for the extent of FHBG uptake. A line profile passing through four cells showed good co-localization of RFP and FHBG. The FHBG substrate displayed no affinity for mammalian TK enzyme. It was observed that wild-type HeLa cells incubated with FHBG (300 μCi, 2 h) showed no measurable radioluminescence signal.

### Example 7

The super-resolution radioluminescence microscopy scheme described in FIG. 4 is aimed at resolving cellular uptake of radiotracer beyond the spatial resolution limit imposed by the physical propagation of the beta particles through the scintillator. To demonstrate a particular embodiment of the technique, a Hamamatsu ImageEM C9100-14 EM-CCD camera was used to acquire 100 ms frames using the maximum gain setting. The samples were placed in a LV200 microscope fitted with a 40× oil lens. A cell culture incubated with FDG was imaged by taking a sequence of 1800 frames. An automated segmentation of the cells was then performed. To confirm the radioluminescence signal, a 5 minute exposure image was also taken.

One of the 100 ms frames was evaluated as an example. Basic image processing with a Gaussian filter helped to resolve weak beta decay signals. Individual beta decay tracks were segmented with an automated algorithm and analyzed with a custom algorithm to estimate where each beta particle was emitted.

After all the beta tracks had been segmented, analyzed and localized, a synthetic image was formed by placing a dot at the location of each presumed beta emission. Such an image is the super-resolution image, in which the blurring inherent to the physical propagation of the energetic betas has been removed.

It was shown that the super-resolution image could be obtained without any prior information or using the prior knowledge of the cell boundaries. In this case, a segmented mask obtained from the brightfield image was used to favor the localization of beta decays near a cell. In addition, the super-resolution FDG image was fused over the brightfield image using the color red and a green color corresponds to GFP fluorescent emission was used. This demonstrated that the super-resolution radioluminescence microscopy scheme can be used concurrently with standard fluorescence microscopy.

From the discussion above it will be appreciated that the invention can be embodied in various ways, including the following:

1. A method for imaging the distribution of radiolabeled molecules in individual cells, comprising: incubating cells with radiolabeled molecules; placing the incubated cells in an imaging device; and imaging scintillation light from individual cells.

2. The method of embodiment 1, further comprising: measuring the distribution of radiolabeled molecules inside, bound to, or surrounding individual cells from the images.

3. The method of embodiment 1, wherein the imaging scintillation light from individual cells comprises: acquiring a sequence of short duration frames of cells; segmenting radiation decay tracks within each frame; localizing individual radioactive decay locations; and generating a synthetic image from the frames.

4. The method of embodiment 1, further comprising: growing cells on a scintillator plate immersed in a cell culture medium; introducing radiolabeled molecules into the cell culture medium and incubating the cells; placing the scintillator plate in an imaging dish; and imaging the scintillation light produced by individual cells from radiolabeled molecules inside, bound to, or surrounding the cells.

5. The method of embodiment 4, wherein the cells are grown sparsely on the scintillator plate to facilitate the imaging and measurement of single cells.

6. The method of embodiment 1, further comprising: growing cells on a scintillator plate immersed in a cell culture medium; placing the scintillator plate in an imaging dish; varying the concentration of radiolabeled molecules in the cell medium; and imaging the scintillation light from radiolabeled molecules inside, bound to, or surrounding the cells; wherein the cells are alive and respond to the varying concentration of radiolabeled molecules in the cell medium; and wherein the imaging is performed at multiple time points to measure change over time.

7. The method of embodiment 6, further comprising analyzing pharmacokinetic properties of radiolabeled molecule uptake in individual cells using a compartmental model.

8. The method of embodiment 1, further comprising: injecting a living subject with radiolabeled molecules; harvesting a tissue of interest from the living subject; placing a scintillator plate in close proximity to the harvested tissue and imaging scintillation light from radiolabeled molecules inside, bound to, or surrounding the tissue cells.

9. The method of embodiment 8, further comprising: dissociating the cells of the harvested tissue; placing the dissociated cells sparsely on the scintillator plate; and imaging scintillation light from radiolabeled molecules inside, bound to, or surrounding the dissociated cells.

10. The method of embodiment 1, further comprising: incubating cells with fluorophore labeled molecules; and imaging fluorescence and scintillation light from the cells.

11. A method for imaging radiolabeled molecules and fluorophore labeled molecules in individual cells, comprising: selecting a first molecule with a first biological activity; selecting a second molecule with a second biological activity; labeling a plurality of the first molecule with a radioactive label; labeling a plurality of the second molecule with a fluorophore label; incubating cells with radioactive labeled molecules and fluorophore labeled molecules; placing the incubated cells in an imaging device; imaging fluorescence and scintillation light from the cells; and analyzing the images.

12. The method of embodiment 11, further comprising: correlating the first biological activity of the first molecule with the second biological activity of the second molecule.

13. The method of embodiment 11, wherein the imaging of scintillation light comprises: acquiring a sequence of short duration frames of cells; segmenting radiation decay tracks within each frame; localizing individual radioactive decay locations; and generating a synthetic image from the frames.

14. The method of embodiment 13, further comprising: fusing the synthetic image with a fluorescent or brightfield image the cells.

15. The method of embodiment 13, wherein the segmenting comprises:

filtering each frame with a Gaussian kernel; transforming the filtered frames with an H-maxima Transform; and thresholding the transformed frame to produce a binary image.

16. The method of embodiment 13, wherein the localization of radioactive decay location comprises: maximizing an optical signal; identifying cells in closest proximity to the optical signal; and disregarding tracks with optical intensity below a threshold intensity.

17. The method of embodiment 11, further comprising: selecting a third molecule with a third biological activity; labeling a plurality of the third molecule with a second type of fluorophore label; and correlating the biological activity of the first molecule, the second molecule and the third molecule.

18. A radioluminescence microscopy system, comprising: an imaging dish configured to hold cells incubated with radiolabeled molecules and cell culture media; a scintillator plate disposed adjacent to the cells; and a microscope, comprising: a stage configured to hold the imaging dish; one or more objective lenses configured to magnify cells within the imaging dish; and an image recording device to record images from the objective lenses; wherein scintillation light is produced by decay of radiolabeled molecules inside, bound to, or surrounding the cells; and wherein the scintillation light is recorded by the image recording device.

19. The system of embodiment 18, wherein the stage of the radioluminescence microscope further comprises: a magnet or a magnetic coil configured to produce a magnetic field in the scintillator plate; wherein the magnet field is oriented orthogonally to the plane of the scintillator plate.

20. The system of embodiment 18, wherein the image recording device is a cooled charge-coupled device (CCD) camera.

21. The system of embodiment 20, wherein the image recording device further comprises electron multiplication gain or image intensification.

22. The system of embodiment 18, wherein the microscope, further comprises: a set of emission filters operably coupled to the image recording device; an excitation light source; and a set of emission and excitation filters;

wherein fluorescence, bioluminescence or brightfield microscopy can be performed concurrently with radioluminescence microscopy.

23. The system of embodiment 18, wherein the scintillator plate has a thickness of approximately 100 μm or less.

24. The system of embodiment 18, wherein the scintillator plate comprises: a layer of scintillator material attached to an interior bottom surface of the imaging dish with a layer thickness within the range of approximately 1 μm and approximately 10 μm.

25. The system of embodiment 24, wherein the imaging dish has a bottom surface with a bottom wall thickness of approximately 100 μm.

26. The system of embodiment 18, wherein the imaging dish is fabricated from a scintillator material with a bottom wall thickness of 100 μm or less.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method for imaging the distribution of radiolabeled molecules in individual cells, comprising:
incubating cells with radiolabeled molecules;
placing the incubated cells in an imaging device; and
imaging scintillation light from individual cells.

2. A method as recited in claim 1, further comprising:
measuring the distribution of radiolabeled molecules inside, bound to, or surrounding individual cells from said images.

3. A method as recited in claim 1, wherein said imaging of scintillation light from individual cells comprises:
acquiring a sequence of short duration frames of cells;
segmenting radiation decay tracks within each frame;
localizing individual radioactive decay locations; and
generating a synthetic image from the frames.

4. A method as recited in claim 1, further comprising:
growing cells on a scintillator plate immersed in a cell culture medium;
introducing radiolabeled molecules into the cell culture medium and incubating the cells;
placing the scintillator plate in an imaging dish; and
imaging scintillation light produced by individual cells from radiolabeled molecules inside, bound to, or surrounding the cells.

5. A method as recited in claim 4, wherein said cells are grown sparsely on the scintillator plate to facilitate imaging of single cells.

6. A method as recited in claim 1, further comprising:
growing cells on a scintillator plate immersed in a cell culture medium;
placing the scintillator plate in an imaging dish;
varying the concentration of radiolabeled molecules in the cell medium; and
imaging the scintillation light from radiolabeled molecules inside, bound to, or surrounding the cells;
wherein the cells are alive and respond to the varying concentration of radiolabeled molecules in the cell medium; and
wherein said imaging is performed at multiple time points to measure change over time.

7. A method as recited in claim 6, further comprising analyzing pharmacokinetic properties of radiolabeled molecule uptake in individual cells using a compartmental model.

8. A method as recited in claim 1, further comprising:
injecting a living subject with radiolabeled molecules;
harvesting a tissue of interest from the living subject;
placing a scintillator plate in close proximity to harvested tissue; and
imaging scintillation light from radiolabeled molecules inside, bound to, or surrounding tissue cells.

9. A method as recited claim 8, further comprising:
dissociating the cells of the harvested tissue;
placing the dissociated cells sparsely on the scintillator plate; and
imaging scintillation light from radiolabeled molecules inside, bound to, or surrounding the dissociated cells.

10. A method as recited in claim 1, further comprising:
incubating cells with fluorophore labeled molecules; and
imaging fluorescence and scintillation light from the cells.

11. A method for imaging radiolabeled molecules and fluorophore labeled molecules in individual cells, comprising:
selecting a first molecule with a first biological activity;
selecting a second molecule with a second biological activity;
labeling a plurality of the first molecule with a radioactive label;
labeling a plurality of the second molecule with a fluorophore label;
incubating cells with radioactive labeled molecules and fluorophore labeled molecules;
placing the incubated cells in an imaging device;
imaging fluorescence and scintillation light from the cells; and
analyzing the images.

12. A method as recited in claim 11, further comprising:
correlating the first biological activity of the first molecule with the second biological activity of the second molecule.

13. A method as recited in claim 11, wherein said imaging of scintillation light comprises:
acquiring a sequence of short duration frames of cells;
segmenting radiation decay tracks within each frame;
localizing individual radioactive decay locations; and
generating a synthetic image from the frames.

14. A method as recited in claim 13, further comprising:
fusing the synthetic image with a fluorescent or brightfield image the cells.

15. A method as recited in claim 13, wherein said segmenting comprises:
filtering each frame with a Gaussian kernel;
transforming the filtered frames with a H-maxima Transform; and
thresholding the transformed frame to produce a binary image.

16. A method as recited in claim 13, wherein said localization of radioactive decay location comprises:
maximizing an optical signal;
identifying cells in closest proximity to the optical signal; and
disregarding tracks with optical intensity below a threshold intensity.

17. A method as recited in claim 11, further comprising:
selecting a third molecule with a third biological activity;
labeling a plurality of the third molecule with a second type of fluorophore label; and
correlating the biological activity of the first molecule, the second molecule and the third molecule.

18. A radioluminescence microscopy system, comprising:
an imaging dish configured to hold cells incubated with radiolabeled molecules and cell culture media;
a scintillator plate disposed adjacent to the cells; and
a microscope, comprising:
a stage configured to hold the imaging dish;
one or more objective lenses configured to magnify cells within the imaging dish; and
an image recording device to record images from the objective lenses;
wherein scintillation light is produced by decay of radiolabeled molecules inside, bound to, or surrounding the cells; and
wherein the scintillation light is recorded by the image recording device.

19. A system as recited in claim 18, wherein the stage of the radioluminescence microscope further comprises:
a magnet or a magnetic coil configured to produce a magnetic field in the scintillator plate;
wherein the magnet field is oriented orthogonally to the plane of the scintillator plate.

20. A system as recited in claim 18, wherein the image recording device is a cooled charge-coupled device (CCD) camera.

21. A system as recited in claim 20, wherein the image recording device further comprises electron multiplication gain or image intensification.

22. A system as recited in claim 18, wherein said microscope, further comprises:
a set of emission filters operably coupled to the image recording device;
an excitation light source; and
a set of emission and excitation filters;
wherein fluorescence, bioluminescence or brightfield microscopy can be performed concurrently with radioluminescence microscopy.

23. A system as recited in claim 18, wherein said scintillator plate has a thickness of approximately 100 μm or less.

24. A system as recited in claim 18, wherein said scintillator plate comprises:
a layer of scintillator material attached to an interior bottom surface of the imaging dish with a layer thickness within the range of approximately 1 μm and approximately 10 μm.

25. A system as recited in claim 24, wherein said imaging dish has a bottom surface with a bottom wall thickness of approximately 100 μm.

26. A system as recited in claim 18, wherein said imaging dish is fabricated from a scintillator material with a bottom wall thickness of 100 μm or less.
